(19) United States
(12) Patent Application Publication
(10) Pub. No.: US 2007/0039626 A1
(43) Pub. Date: Feb. 22, 2007

(54) Device for the intraperitoneal application of a fluid which features ozone or ozonized oxygen into a human or animal body and use therefor

(75) Inventor: Siegfried Schulz, Weimar-Niederwalgen (DE)

Correspondence Address: PYLE & PIONTEK, 221 N. LASALLE - ROOM 2036, CHICAGO, IL, 60601, US

(73) Assignee: Philipps-Universitat Marburg, Marburg (DE)

(21) Appl. No.: 11/544,979

(22) Filed: Oct. 5, 2006

Related U.S. Application Data
(63) Continuation-in-part of application No. PCT/DE05/00587, filed on May 4, 2005.
(63) Continuation-in-part of application No. PCT/DE05/00597, filed on Apr. 5, 2004.

(30) Foreign Application Priority Data
Jul. 4, 2004 (DE) 10 2004 017 599.3

(51) Int. Cl. A61B 19/00

(52) U.S. Cl. 128/898; 422/186.07; 604/25; 604/26

(57) ABSTRACT

The subject matter of the invention is the use of a device for the application of a fluid which features ozone or ozonized oxygen into a human or animal body and a device for this. The creation of a use and device which use the beneficial effects of ozone to combat tumors without causing side effects which are damaging to someone's health is achieved by applying the fluid intraperitoneally.

This application is a continuation-in-part of PCT/DE2005/000597, filed Apr. 5, 2004 and claims Priority from German Application No. DE 10 2004 017 599.3 filed on 7. Apr. 2004

### BACKGROUND OF THE INVENTION

1. Field of the Invention

The invention at hand concerns a use of a device for the application of a fluid which features ozone or ozonized oxygen into a human or animal body and a device for the intraperitoneal application of a fluid which features ozone or ozonized oxygen on or in a tumor located in a human or animal body.

2. Description of the Prior Art

It has been scientifically proven that ozone can kill bacteria, therefore ozone or ozonized oxygen is also used for the therapeutic treatment in humans or animals. In the process, an improper application of ozone can also cause damage to someone's health, thus ozone has been used extracorporeally, in body lumens, in the form of ozone-enriched blood or in the form of hyperbaric ozone therapy until now. A device for the use of hyperbaric ozone therapy is known from DE 43 40 730 C2, for example.

It is scientifically proven that the bacterial- and tissue-killing effect of ozone also leads to the death of entire tumors when just enough ozone is applied.

It is understood that metastases, sarcomas, or other growths are classified under tumor, in particular when these are malignant.

### BRIEF SUMMARY OF THE INVENTION

Starting from there, the underlying aim for the invention at hand is to also use the beneficial effects of ozone for combating tumors, without causing side effects damaging to someone's health.

In this, the knowledge that the damaging effects of ozone do not occur or occur to a much lesser extent when the ozone is applied in the immediate vicinity of the tumor or even into the interior of the tumor underlies the invention at hand. Furthermore, the knowledge that the damaging effects of the ozone do not occur or occur to a much lesser extent when the ozone or ozonized oxygen is applied with minimal pressure also underlies the invention at hand.

According to the invention at hand, the use of a device for the application of a fluid which features ozone or ozonized oxygen into a human or animal body according to the characteristics of claim 1 and a device according to the characteristics of claim 7 are proposed as a technical solution to reach the aim. Advantageous further developments of this method and this device are described in the respective sub-claims.

A method developed further according to this technical knowledge and a device formed according to this technical knowledge have the advantage that the ozone or ozonized oxygen comes directly into contact with the tumor through the intraperitoneal application (application in the abdominal cavities) of the fluid and can thus directly develop its effect. This effect is even strengthened when the fluid is applied in the immediate vicinity of the tumor or even in the tumor itself.

In a preferred application, the fluid is applied with a very low pressure between 20 mbar and 50 mbar. This has the advantage that the fluid comes very softly into contact with both the diseased as well as the healthy tissue. Surprisingly and contrary to the previously prevailing opinion, no health-damaging side effects occurred in laboratory animals despite intensive and prolonged application.

In yet another use, up to 100 ml, preferably 80 ml, per kg body weight was applied without the occurrence of side effects which must be taken seriously. In this, the fluid was advantageously applied in intervals, in particular in 3 units spread throughout the day.

The embodiment of a corresponding device with a low working pressure of 20 mbar to 60 mbar and a precision manometer eases the treatment for the attending physician, since he can apply the fluid very easily and comfortably with the desired pressure. A further advantage exists in that an exact and individual adjustment of the pressure corresponding to the patient is possible due to the precision manometer. Yet another advantage exists in that exact control of the actual pressure conditions can also be accomplished, in order to optimally monitor the treatment.

Security devices, which prevent an accidental application with an undesired pressure, have been created by the warning function and, in particular, by the interrupting mechanism. Consequently the safety of the treatment is increased through this.

In a particularly preferred embodiment, a potentiometer is provided for zero adjustment. This has the advantage that atmospheric pressure variations can be compensated hereby, in order to allow precise pressure adjustment and pressure control.

Further advantages of the use according to the present invention and the device according to the present invention result from the practical embodiments described in the following. Likewise the aforementioned characteristics, which are further described in the following, can be used according to the present invention alone or in any combination with one another, respectively. The practical embodiments mentioned are not to be understood as an exclusive list, but rather have an exemplary character.

### BRIEF DESCRIPTION OF THE DRAWING

FIG. 1 of the drawing shows a schematic depiction of the device for the intraperitoneal application of a fluid according to the present invention.

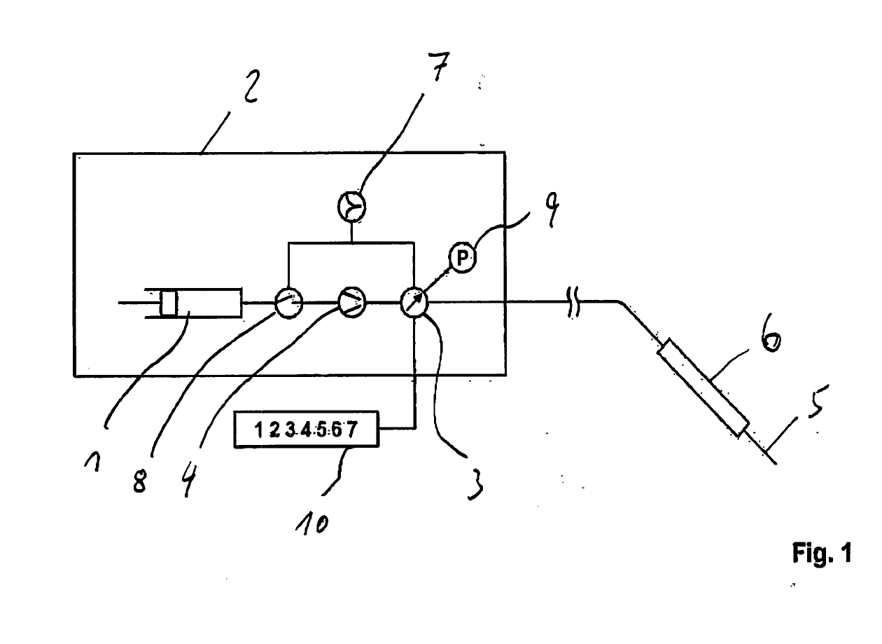

### DETAILED DESCRIPTION OF THE INVENTION

In a practical embodiment as shown in the drawing, an approx. 8 l fluid storage container 1 realized as a piston cylinder is situated in a case 2 together with a precision manometer 3. Furthermore, a pump 4 is provided in the case 2, which pumps the fluid out of the fluid storage container 1 into a cannula 5 with a pressure between 20 mbar and 60 mbar. This cannula 5 is suitable for intraperitoneal application and equipped with an endoscope 6, so that the cannula 5 can be guided precisely in the abdominal cavity and led to the tumor.

Furthermore, a warning device 7 is provided in the case 2, which emits an acoustic, visual, or electronic signal as soon as the working pressure exceeds 40 mbar. If this is the case, then the attending physician can undertake an inspection of the treatment. If the working pressure exceeds 50 mbar, then the interrupting mechanism 8 automatically switches the supply of fluid off, in order to avoid damage to the health of the patient.

A potentiometer 9 is connected to the precision manometer 3, in order to measure the atmospheric pressure before the start of the treatment and to adjust the precision manometer 3 to the atmospheric pressure. Through this, an exact and constant output of the fluid occurs.

For this, it has been proven to be advantageous to make the current pressure easily visible on a display 10.

What is claimed is:

1. A method for supplying a fluid containing ozone or ozonized oxygen into a human or animal body, comprising the step of applying the fluid intraperitoneally.

2. The method of claim 1 wherein the fluid is applied in the immediate vicinity of a tumor.

3. The method of claim 1 wherein the fluid is applied in the interior of the tumor.

4. The method of claim 1 wherein the fluid is applied with a pressure of 20 mbar to 50 mbar.

5. The method of claim 1 wherein the fluid is applied in an amount up to 100 ml, preferably 80 ml, per kg of the body weight of the human or animal.

6. The method of claim 1 including the steps of applying the fluid in intervals.

7. Device for the intraperitoneal application of a fluid which contains ozone or ozonized oxygen on or in a tumor located in a human or animal body, comprising: a fluid storage container, a device connected to the fluid storage container for the delivery of the fluid from the fluid storage container into a body with a working pressure of 20 mbar to 60 mbar and with a precision manometer having a resolution between 0.1 mbar and 3 mbar for measuring the current pressure.

8. The device of claim 7 including a warning device that emits a warning signal at a working pressure of 40 mbar or more.

9. The device of claim 7 including an interrupting mechanism that stops the release of the fluid at a working pressure of 50 mbar or more.

10. The device of claim 7 wherein the fluid storage container holds at least 5 liters of the fluid.

11. The device of claim 7 wherein the storage device is cylinder is a piston cylinder mechanism.

12. The device of claim 7 including a potentiometer connected to the precision manometer for making a zero adjustment of the precision manometer.

13. The device of claim 7 including an intraperitoneal cannula.

14. The device of claim 13 including an endoscope connected to the cannula.

15. The device of claim 7 including a display for the visual identification of the current working pressure.

16. The device of claim 7 wherein the precision manometer has a resolution of 1 mbar.

17. The device of claim 7 wherein the fluid storage container holds at least 8 liters of the fluid.
